(19) United States
(12) Patent Application Publication
(10) Pub. No.: US 2026/0232956 A1
(43) Pub. Date: Aug. 13, 2026

(54) CATHETER KIT

(71) Applicant: Clinisupplies Limited, Watford (GB)

(72) Inventors: Jeston D'Costa, Watford (GB); Martin Beynon, Watford (GB)

(21) Appl. No.: 19/150,787

(22) PCT Filed: Jan. 23, 2024
(86) PCT No.: PCT/EP24/51566

§ 371 Date: Jul. 24, 2025

(30) Foreign Application Priority Data
Jan. 25, 2023 (GB) 2301081.2

(51) Int. Cl. A61M 25/00

(52) U.S. Cl. CPC A61M 25/002; A61M 25/0097; A61M 2202/0496; A61M 2205/586

(57) ABSTRACT

The invention provides a kit comprising: i) a catheter, ii) a catheter casing, and iii) a handle; wherein the catheter comprises a tube and a funnel, wherein the funnel comprises a handle-engaging portion; and the handle is configured to engage with the handle-engaging portion of the catheter funnel. Methods of using the kits to insert a urinary catheter are also provided.

### INTRODUCTION

The present invention relates to kits comprising catheters for intermittent catheterisation and methods of using such kits.

### BACKGROUND

Intermittent self-catheterisation is a process used to help patients urinate when they are unable to. The need for self-catheterisation may arise in patients who have difficulty emptying their bladder for a number of reasons.

It may be short term, for example in patients who have undergone bladder surgery or continence surgery to stop leakage of urine.

Alternatively, the need for self-catheterisation may be long-term in patients who suffer or have suffered from central nervous system tumours, an enlarged prostate, multiple sclerosis, neurogenic bladder, strokes, urinary incontinence, urinary retention or urethral strictures.

In some patients the process of intermittent catheterisation may be carried out by a caregiver, for example if the patient is vulnerable because of age or disability. In most cases, where the patient is able, intermittent catheterisation is carried out by the patient themselves, hence it is termed intermittent self-catheterisation.

The process involves inserting a flexible tube, referred to as a catheter, into the urethra until it reaches the bladder. Urine can then drain out of the bladder through the catheter and the urine can be collected either into a toilet, collection bag or container. Once the bladder is emptied the catheter is removed.

The process of intermittent catheterisation is usually repeated at regular intervals throughout the day, for example every 4 to 6 hours. The process should be repeated often enough so that the user's bladder does not fill over 500 ml and the user should adjust how often the process is repeated depending on their fluid intake each day.

Although many types of intermittent catheters already exist, many of these are difficult to handle and insert. This is in part due to the fact that catheters are often lubricated to assist insertion. However, a result of this is that they are difficult and slippery to handle.

In addition, self-catheterisation may give rise to risks of urinary infections, as a result of bacteria transferring from the user's hands to the catheter before insertion. Patients are advised to clean their hands thoroughly before insertion and switch to a new catheter as required. Typically, intermittent catheters are single use, so patients should only use a catheter one time before throwing it away and replacing it with a new catheter, however there is still a high risk of infection.

An example of an existing catheter kit is the LoFric® Elle catheter, which is described in WO 2019/224263. This kit consists of a catheter tube and catheter casing, wherein the catheter tube is connected to the lid of the catheter casing. It functions so that when the lid is pulled off the body of the catheter casing the catheter slides out of the body of the casing and the body can then be attached to the lid, at an angle perpendicular to the catheter tube, to function as a handle for insertion of the catheter.

The main problem with this type of catheter kit is that the catheter casing cannot be stood in a stable upright position without the user holding it. Therefore, if the user needs to place the catheter down before inserting it, such as while they prepare for catheterisation (for example, whilst they are washing their hands) there is no clean, secure place for them to put the catheter, resulting in a high risk of infection.

Another problem with this type of catheter kit is that, because the lid of the casing also functions as the funnel of the catheter, the user must grip on to the funnel while they remove the casing and attach the handle. Because the funnel/lid comes into such close proximity with the user's body, this results in a further risk of infection.

Further, another problem with this type of catheter kit is that, because the body of the catheter casing also functions as the handle, the catheter tube cannot be placed back into the casing while the handle is in use. One reason why the user may wish to put the catheter down before inserting it, for example, would be to prepare themselves for catherisation, before removing the catheter from the casing and attaching the handle. Alternatively, the user may just want to place the catheter down briefly whilst they adjust their grip or adjust the attachment of the handle to the catheter. Self-catheterisation can be tricky and it is common for the user to adjust their grip while attempting to insert the catheter, therefore having no secure casing to place the catheter back into while the handle is in use makes catheter insertion difficult. Also, because lubricant is typically used for catheter insertion, the catheter and handle can get very slippery, making it easy to drop the catheter, e.g. while adjusting your grip during insertion. If the catheter is dropped there will be a high risk of infection and the user may have to start the process again with a different catheter tube.

There therefore exists the need for improved catheter kits that can be used by patients who need to undergo intermittent self-catheterisation, preferably ones that are easy to handle and reduce the risk of infection.

### SUMMARY OF THE INVENTION

The invention provides kits comprising catheters for intermittent catheterisation and methods of using such kits.

Accordingly, in a first aspect, the invention provides a kit comprising:
i) a catheter;
ii) a catheter casing; and
iii) a handle;
wherein the catheter comprises a tube and a funnel, wherein the funnel comprises a handle-engaging portion;
and the handle is configured to engage with the handle-engaging portion of the catheter funnel.

The kit may comprise at least three separate components, namely a catheter, a catheter casing and a handle. Suitably, the kit comprises four separate components, namely a catheter, a catheter casing body, a catheter casing lid and a handle.

The handle can be used for engagement with the catheter funnel, such that when the handle is lifted the catheter can be removed from the catheter casing to allow the user to use the catheter (i.e. remove the catheter from the casing and insert it into their body and/or remove the catheter from their body and insert it into the casing), without touching it with their hand(s). This reduces the risk of contamination of the catheter from pathogens that may have been picked up by the user on their hands.

As mentioned above, the handle may be a separate component of the catheter kit, i.e. it may be separate from the catheter and the catheter casing, Accordingly, the handle may be removably connectable to the catheter funnel, not fixedly connected to the catheter funnel. For example, the handle may be configured to engage with the catheter funnel and subsequently disengage with the catheter funnel.

The catheter comprises a catheter tube and a funnel. The catheter may be a hydrophilic catheter, i.e. the catheter tube has a hydrophilic coating, which provides a lubricious surface when hydrated with water. Alternatively, the catheter may not be provided by a hydrophilic coating and a separate lubricant is present.

Catheters are well-known and typically comprise a tube and a funnel, wherein the tube functions as a conduit to pass the urine from the bladder to outside of the body. Accordingly, the tube may be made from a flexible material. Typically, the tube is made from soft but durable plastic, such as a polymer, rubber or silicone. Preferably the tube is made from medical grade polymer, such as polyvinyl chloride or thermoplastic polyurethane. The tube may be between 8 cm and 20 cm long, preferably between 8 cm and 10 cm long. The diameter of the tube is typically measured in Charrière, where 1Ch is equal to ⅓ mm. The tube may have an external diameter of 8Ch to 20Ch, preferably 10Ch to 16Ch. The tube may have an internal diameter of 6Ch to 18Ch, preferably 8Ch to 12Ch.

As will be accepted, the dimensions of the catheter tube may vary depending on patient suitability. For example, paediatric patients require smaller sized catheters. A paediatric catheter may suitably have an external diameter of 6Ch to 8Ch.

The funnel can have multiple functions. It may be used to hold or attach a handle to when handling the catheter. It can also be used to help guide the urine into the toilet or even to attach a urine bag to enable self-catheterisation when a bathroom is not available or when the user needs to measure urine output. Alternatively, the funnel may be used to connect a catheter extension tube to the catheter, this use of the funnel may be employed by wheelchair users who require an extended catheter to ensure the length of the tube reaches the toilet. The funnel, or at least a section of the funnel, may be concave in shape, such that it has a reduced diameter in the middle compared to its ends. This enables the handle to remain securely connected to the funnel, without the funnel sliding through the funnel-engaging portion of the handle.

Preferably, the catheters in the kits of the present invention are female catheters. Female catheters are generally distinguished from male catheters in that they are shorter in length than male catheters. Male catheters are typically around 40 cm in length, whereas female catheters are typically only around 15 cm to 20 cm or shorter in length.

Catheter kits of the invention may provide a catheter casing wherein the casing comprises a body having a hollow passage and a lid removably connectable to the body. The casing is typically made of thermoplastics, preferably polypropylene.

The lid of the catheter casing may be detachable and re-attachable to the body of the casing. Suitably, the lid may be connected to the body and a releasable seal may be formed between the lid and the body, such that the catheter is contained or sealed within the casing. Next, the lid may be removed from the body and the seal may be released, allowing the catheter to be removed from the casing. Then, the lid may be re-connected or re-attached to the body and the seal between the lid and the body may be re-formed. Accordingly, both the lid and the body of the casing may be reusable.

Typically, the lid may be removable from the body by pulling it away from the body and re-attachable by pushing it towards the body. For example, the lid may be connectable to the body via a click-fit mechanism, i.e. the lid may click into place/onto the body. Alternatively, the lid may be removeable from the body by twisting the lid in one direction and re-attachable to the body by twisting the lid in the opposite direction. For example, the lid may be connectable to the body via a screw-fit mechanism. In one example, the casing may comprise a screw cap thread and the lid may screw or twist onto the body.

The interior of the lid may be dimensioned to accommodate the bottom of the catheter body, such that the lid can act as a stand for the body. In this case, the catheter casing may be stood up in an upright position on a flat surface, to allow the user to place the casing down (e.g. while they get the handle out of their bag) without the risk of the casing falling on its side and the catheter falling out of the hollow passage, resulting in contamination of the catheter.

Alternatively, the user may wish to place the catheter down in an upright position while they wash their hands. The user is therefore able to remove the casing (which is not sterile and may have bacteria on it) from their bag, place it in an upright position, ready for insertion, then wash their hands so that after washing their hands they do not need to touch the catheter casing again. This enables a very limited risk of infection.

In another aspect of the invention the closed end of the body of the casing may comprise a flat bottom surface for standing the casing in an upright position, without the need to stand the body in the upturned lid.

The hollow passage inside the catheter casing may be circular and have a diameter larger than the diameter of the tube but smaller than the diameter of the funnel, such that the catheter tube can be inserted into the hollow passage within the casing but that the funnel cannot. The funnel therefore protrudes out of the hollow passage to allow the user to grip it when removing the catheter from the casing.

The diameter of the hollow passage may be at least 0.5 mm larger than the outer diameter of the catheter tube, or preferably at least 1 mm larger than the outer diameter of the catheter tube or more preferably at least 3 mm larger than the outer diameter of the catheter tube, to allow space for lubricant in the catheter casing such that when the catheter is in the casing there is a layer of lubricant between the catheter tube and the catheter casing. Typically, the catheter casing may contain 1-5 ml of lubricant, preferably it will contain 2-3 ml of lubricant. Alternatively, when the catheter is a hydrophilic-coated catheter, the lubricant may be replaced with water to activate the hydrophilic coating.

The body of the catheter casing may comprise a main portion, with one open end, and an insert which is partially or fully inserted into the hollow passage at the open end of the main portion. The insert may be permanently connected to the casing body. For example, the insert may be welded into place. Alternatively, the insert may not be permanently connected to the casing body. For example, the insert may be connectable to the casing body by a click-fit mechanism. In one example, the insert may be removable from the casing body, although it is not typically intended to be removed during use.

The inner diameter of the insert may be dimensioned such that it is larger than the outer diameter of the catheter tube but smaller than the diameter of the funnel, therefore allowing the insert to function as a bottleneck to stop the funnel going into the hollow passage. The insert is also tapered to wipe any excess lubricant off the catheter as it is removed from the casing.

The insert may be a hollow cylindrical shape with an opening at both ends end, such that the catheter tube can be passed through the insert and into the casing body. For example, the insert may be short in length and may be inserted into the open end of the casing body such that it only surrounds a small section of the catheter tube, such as the end of the catheter tube which is in contact with the catheter funnel. The insert may be welded to the casing body to keep it in place.

Alternatively, the insert may be a hollow cylindrical shape with one open end and one closed end. For example, the insert may fit inside the casing body such that the closed end of the insert aligns with the closed end of the casing body and the open end of the insert aligns with the open end of the casing body. The insert may be equal in length to or longer in length than the catheter tube and, hence, it may surround the entire catheter tube. The insert may not be permanently fixed to the casing body, for example it may be connectable to the casing body by a click-fit mechanism. Optionally, the insert may contain liquid or gel, such as lubricating liquid.

The insert may also function to allow the lid to fix on to the body, either by a click-fit or screw-fit mechanism. Fixed in a groove on the outer surface of the insert may be an O-ring which contacts the inner surface of the lid when the lid is fixed on to the body, such that the lubricant inside the casing cannot leak out of the casing. The O-ring is typically made from a resilient elastic material, preferably it is made from nitrile or silicone. The O-ring may act as a seal to ensure a sterile barrier.

The lid typically comprises a hollow internal space which is dimensioned to accommodate the catheter funnel. Accordingly, the lid may be at last 2 cm in length, preferably at least 3 cm in length. The lid may be less than 4 cm in length, preferably less than 4 cm in length. The hollow internal space inside the lid may have a diameter larger than the diameter of the funnel, typically the hollow internal space may be larger than 0.5 cm, preferably the hollow internal space will be larger than 1 cm.

The lid may comprise a flat surface on one side and an open end on an opposing side of the flat surface, which forms the hollow internal space, wherein the open end is dimensioned in order to receive the bottom of the catheter casing body, such that the catheter casing body can be placed inside the lid to support the catheter casing without the need for the user to hold it.

The funnel may be made from a flexible plastic. Typically, the funnel may be made from polyvinyl chloride. The funnel may comprise an upper part and a lower part. The upper part may be concave or straight (meaning that it has a constant diameter along its length) and it may comprise a handle engaging portion for engagement with the handle of the catheter kit, preferably for removable engagement with the handle of the catheter kit. The handle engaging portion may be positioned on the upper part of the catheter funnel or it may be positioned on the lower part of the catheter funnel. Alternatively, the handle-engaging portion may constitute the entire catheter funnel, so that the handle may engage with any part of the funnel. Typically, the handle engaging portion of the catheter funnel may be either flat or concave and shaped to limit movement of the handle up and down the length of the funnel whilst it is engaged with the funnel. The handle-engaging portion of the catheter funnel may be formed from a resiliently biased material. Preferably, this will be the upper part of the funnel. Typically, there may be a part of the funnel which does not engage with the handle, this part may be convex and it may be ribbed to aid the users grip of the funnel. Preferably, this will be the lower part of the funnel.

The handle may engage with the catheter funnel at an angle with respect to an axis through the centre of the catheter tube. Hence, when engaged with the catheter funnel the handle may be angularly displaced with respect to the axis through the centre of the catheter tube. For example, the handle may engage with the catheter funnel at an angle of between 45° and 135°, or preferably at an angle of between 70° or 110°, or more preferably at an angle of between 80° and 100°, with respect to the catheter tube. Suitably, the handle may engage with the catheter funnel at a right-angle or 90° angle with respect to an axis through the centre of the catheter tube, such that when engaged the handle and the catheter tube are perpendicular to each other.

Typically, the handle may comprise a shaft and a pair of jaws at one end of the shaft to grip the handle-engaging portion of the catheter funnel, such that the handle can be used to remove the catheter from the casing for use or replace the catheter in the casing for safe disposal of the used catheter. The jaws are typically non-moveable, i.e. they are fixed with respect to the handle, and extend from the shaft. The jaws are typically dimensioned to form a substantially semi-circular opening in the end of the handle, which can engage with the funnel. The shaft of the handle may be provided with one or more protuberances and/or textured surfaces to make the shaft easier to grip.

As will be appreciated, both the catheter and the catheter casing are typically single use products. However, the handle can be easily sanitised and does not need to be replaced regularly. Accordingly, the invention also provides a kit comprising a handle and two or more catheter casings, each containing a catheter, wherein the handle, catheter casings and catheter are as defined herein. Typically, catheter kits of the invention may have one handle and ten or more catheter casings, each containing a catheter. Preferably, catheter kits of the invention may have one handle and twenty or more catheter casings, each containing a catheter. The catheter kits may also include sanitising/anti-bacterial wipes to enable the user to clean the reusable handle between use.

Users of the catheter kits of the invention may carry more than one catheter and catheter casing on them (i.e. in their bag) at any given time. Therefore, there is a need for a marker which flags to the user which catheters have been used and which haven't. Thus, catheter kits of the invention may further comprise an anti-tamper sticker which is removed or disrupted when the catheter casing is opened. The anti-tamper sticker may be positioned between the lid and the body of the catheter casing.

The invention also provides a method of inserting a catheter, the method comprising:
i) providing a kit as described herein.
ii) engaging the handle with the catheter funnel;
iii) removing the catheter from the catheter casing and inserting the catheter tube into a person (e.g. into the urethra of a patient).

The method may also include a step involving removing the lid from the casing, upturning the lid and placing the upturned lid on a flat surface, followed by standing the body of the casing in the upturned lid.

The method may also include a step involving using the handle to remove the catheter tube from the user after use, replacing the catheter into the hollow passage of the catheter casing and detaching the handle from the funnel. The method may further include a final step wherein the lid of the casing is placed back onto the body and the casing is closed, for safe disposal of the catheter.

The method may further include an intermediate step wherein an anti-tamper sticker is removed or disrupted upon opening the catheter casing.

### BRIEF DESCRIPTION OF THE DRAWINGS

FIG. 1 shows an exploded view of a catheter kit according to one embodiment of the invention.

FIG. 2 shows a catheter kit according to one embodiment of the invention, wherein the catheter is inside the body of the catheter casing, wherein the lid of the catheter casing is being used as a stand to hold the catheter casing in an upright position and wherein the handle is attached to the handle-engaging portion of the catheter funnel.

FIG. 3 shows a catheter kit according to one embodiment of the invention, wherein the catheter is inside the catheter casing and wherein the handle is attached to the handle-engaging portion of the catheter funnel.

FIG. 4 shows the catheter casing comprising a body and a lid.

FIG. 5 shows a catheter comprising a tube and a funnel.

FIG. 6 shows a handle for attachment to the handle engaging portion of the catheter funnel.

FIG. 7 shows an exploded view of a schematic of a catheter kit according to an embodiment of the invention.

FIGS. 8 and 9 show schematics of the body of a catheter casing.

FIGS. 10 and 11 show schematics of the insert of a catheter casing.

FIGS. 12 and 13 show schematics of a handle for attachment to the handle engaging portion of the catheter funnel.

FIGS. 14 and 15 show schematics of the lid of a catheter casing.

FIGS. 16 and 17 show schematics of a catheter funnel.

### DETAILED DESCRIPTION OF THE INVENTION

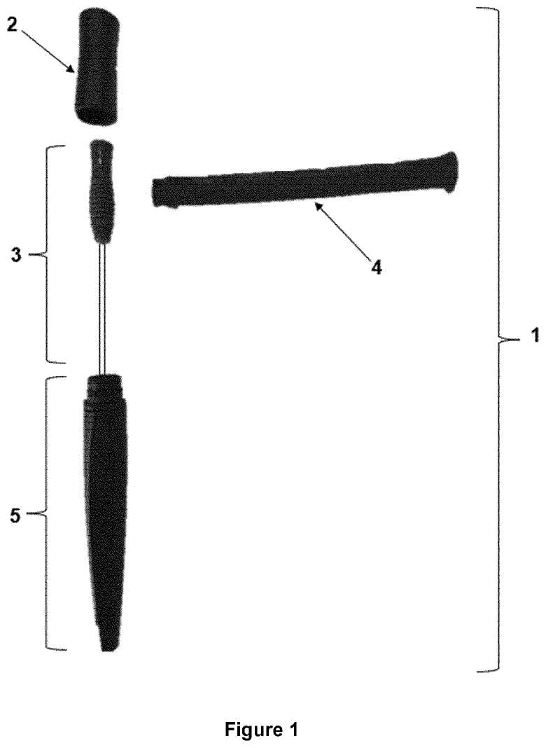

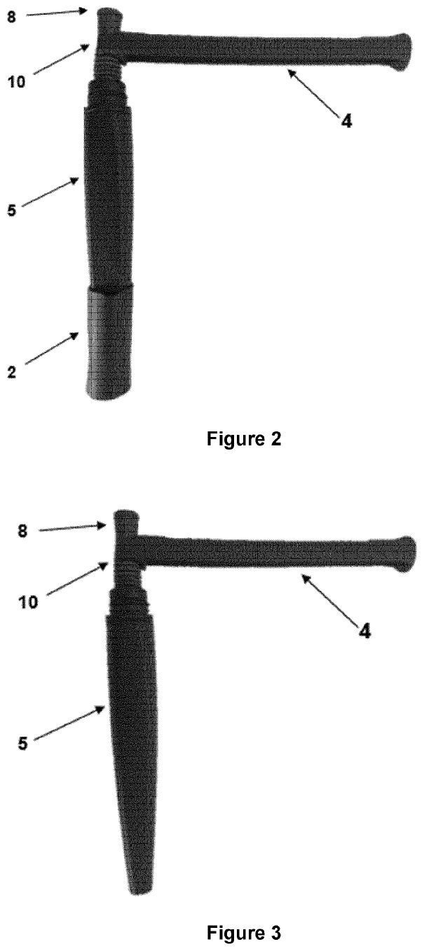

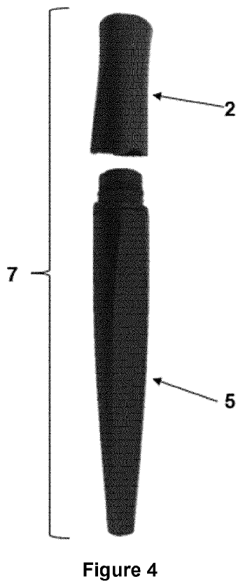

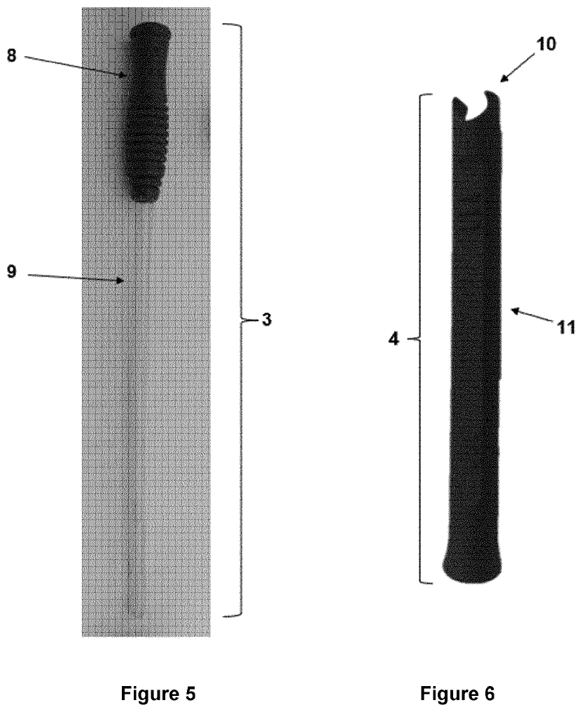

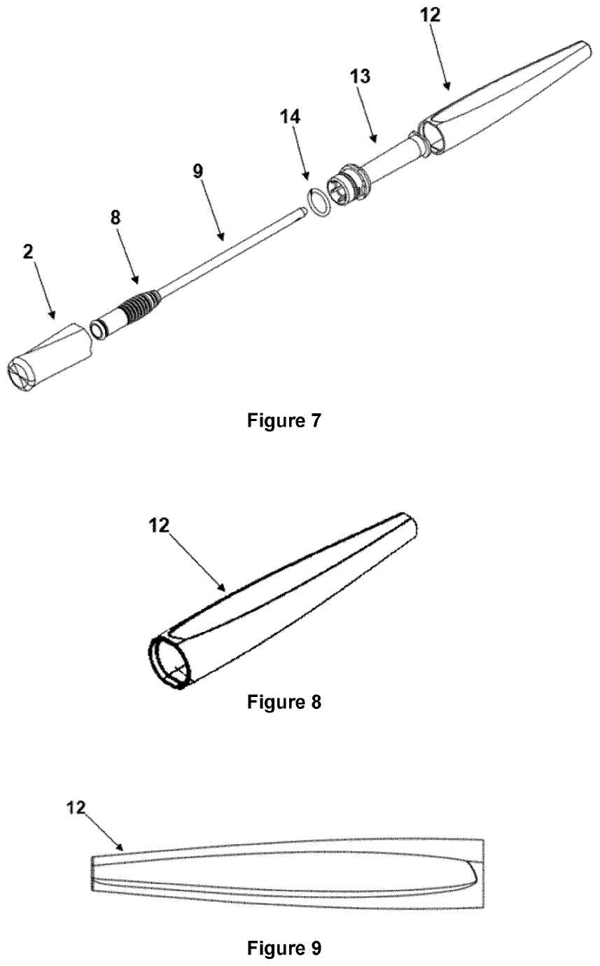

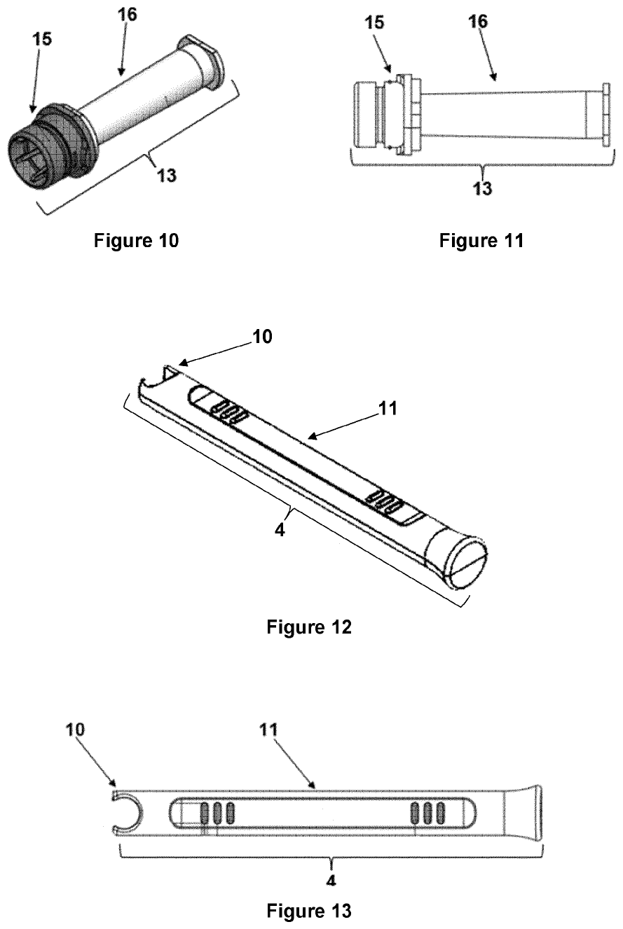

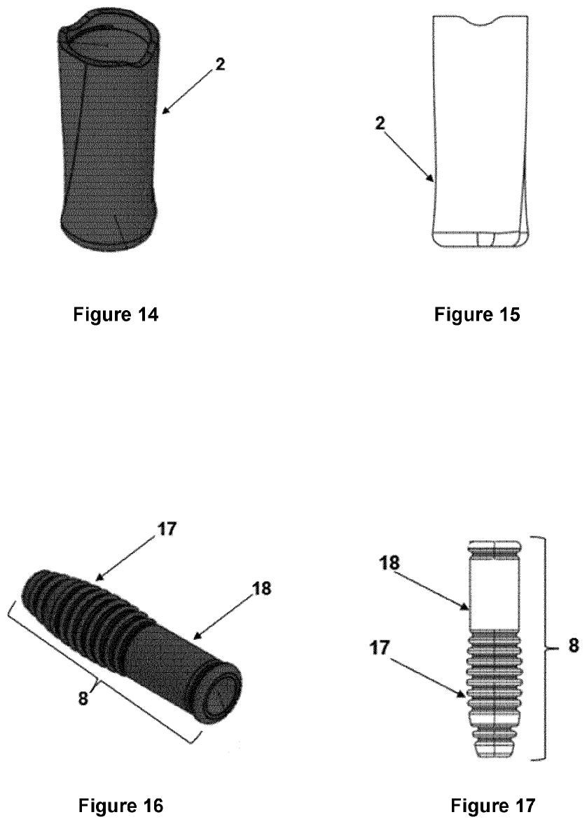

As shown in FIG. 1, the invention provides a catheter kit (1).

The kit (1) comprises a catheter (3), a catheter casing (7), wherein the casing (7) comprises a body (5) and a lid (2), and a handle (4).

The catheter (3) comprises a flexible transparent tube (9) formed from polyvinylchloride. The tube (9) has an external diameter of 12Ch and a length of approximately 9 cm.

One end of the catheter tube (9) is provided with a funnel (8) formed from polyvinylchloride or thermoplastic polyurethane. The funnel (8) is hollow and at one end forms a friction-fit fluid tight seal with an end of the catheter tube (9). The lower part of the funnel (8) (i.e. the part connected to the tube (9)) is externally ribbed. The ribbed surface functions to aid the users grip of the funnel (8) in case any of the lubricant gets onto the funnel making it slippery to handle. The upper part of the funnel (8) is not ribbed and has a concave profile.

The handle (4) is a single-piece moulded component formed from polypropylene. One end of the handle (4) is provided with a pair of rigid jaws (10), which are dimensioned to engage with the upper part of the funnel (8). The jaw opening (i.e. the distance between the two proximal ends of the jaws (10), the proximal ends being the outermost ends) are slightly smaller than the narrowest diameter of the upper part of the funnel (8). When force is applied to insert the funnel (8) into the jaws (10), the funnel (8) deforms so that it may enter the jaws (10). Once inserted into the jaws (10), the funnel (8) can revert to its usual shape such that it is held within the jaws (10). Likewise, the funnel (8) can deform to allow it to be removed from the jaws (10) when sufficient force is applied.

The body (11) of the handle (4) has a generally cylindrical profile with a flattened section to improve the user's grip of the handle (4). To further improve grip, the flattened section is provided with ridges. The end of the handle (4) opposite the jaws (10) has an enlarged diameter to prevent the handle (4) from slipping through the user's hand when gripped.

The casing (7) comprises a lid (2) and a body (5), wherein the body (5) comprises a main portion (12), an insert (13) and an O-ring (14), the insert (13) having a top section (15) and an internal part (16). Both the lid (2) and body (5) are formed form polypropylene.

The lid (2) has a substantially cylindrical body with a flat wall at its top to allow it to stand upturned on a flat surface.

The body (5) comprises a circular hollow passage which is closed at its lower end by virtue of the bottom of the body but is open at its top end to allow the catheter tube (9) to be inserted into the hollow passage. The length of the hollow passage (and therefore the length of the casing body) is at least as long as the length of the catheter tube (9) such that the catheter tube (9) can be fully inserted into the hollow passage. Similarly, the diameter of the hollow passage is at least as large as the external diameter of the catheter tube (9).

The hollow passage may be filled with a lubricating liquid in order to lubricate the catheter tube (9) while it is stored in the casing (7), such that the catheter (3) is lubricated and ready for use. Accordingly, the diameter of the hollow passage may be slightly larger than the external diameter of the catheter tube to accommodate for space for the lubricant between the external wall of the catheter tube and the internal wall of the body of the casing. The lubricant contained within the catheter casing may be a water-soluble lubricant, such as urethral lubricant gel.

At the open end of the hollow passage there is an insert (13), which may be provided with a ledge which may be formed by a section having a reduced diameter (i.e. smaller than the diameter of the hollow passage, but larger than the external diameter of the catheter tubing). The ledge serves to scrape excess lubricant off the catheter tube (9) as it is removed from the casing (7) and also provide a seat for the catheter funnel (8) while the catheter (3) is located in the casing (7).

The exterior of the closed end of the body (5) of the catheter casing may be tapered such that it narrows towards its end. As a result of this the tapered end of the catheter casing can be placed inside the upturned lid (2) to support the body (5) of the catheter casing, without the user needing to hold it.

Alternatively, in another embodiment of the invention, the exterior of the closed end of the body (5) of the catheter casing may not be tapered such that it narrows towards its end. Instead, the diameter of the body (5) of the catheter casing may be constant throughout its entire length and the bottom surface (i.e. the closed end of the body of the catheter casing) of the body may be flat. In this embodiment, the body (5) of the casing may remain in an upright position on a flat surface, without the user needing to hold it or the body (5) needing to be placed inside the upturned lid (2).

The insert (13) fits onto the main portion (12) of the body (5) such that the internal part (16) sits within the main portion (12) and the top section (15) protrudes out of the main portion (12). Within the top section (15) of the insert (13), surrounding and upstanding from the ledge, is a circular wall. The circular wall is dimensioned to engage in a friction-fit with the interior of the lid (2). There may be a small, raised contour or bump on either side of the outer surface of the circular wall which engages with either side of the inside wall of the lid to keep it securely fastened. The circular wall may also be provided with a circumferential recess in which the rubber O-ring (14) is located, which provides a seal with the lid (2) (when attached to the body (5)) to prevent any lubricant from leaking out of the casing (7) and to ensure a sterile environment within the casing. The bottom edge of the lid (2) is provided with two curved cut-outs on opposing sides, which can align with complementarily shaped curved protrusions external to the upstanding wall. The arrangement allows the lid (2) to be removed from the body (5) using a twisting motion.

In use, the kit (1) is provided with the catheter tube (9) located within the catheter casing (7) (with the lid (2) secured to the body (5), optionally having an anti-tamper sticker between the lid (2) and body (5)). To open the casing (7), the lid (2) can be twisted and pulled away from the body (5) to expose the catheter funnel (8). Upon opening the casing, the anti-tamper sticker will be removed or disrupted. The user can then take the handle (4) and push the jaws (10) of the handle (4) against the concave surface of the catheter funnel (8) such that the jaws (10) engage around the catheter funnel (8). The catheter (3) can then be removed from the body of the casing (5) using the handle (4) and inserted into the user to drain urine from their bladder. Once finished, the user can dispose of the used catheter by replacing the catheter (3) back into the body of the casing (5), pull the handle (4) away to disconnect the jaws (10) from the catheter funnel (8) and replace the lid (2) on to the body (5) of the casing (7).

At all times, the user's hand only touches the catheter casing (7) and the catheter handle (4), rather than the catheter (3) itself, to allow for a reduced risk of contamination of the catheter (3). The handle (4) is reusable and can be easily sanitised between uses.

Both the catheter (3) and catheter casing (7) are single use and should therefore be disposed of and replaced after every use. The handle (4) need not be replaced as this can be easily sanitised and used in the same way to remove and insert the new catheter (3) from a new catheter casing (7).

1. A kit comprising:
i) a catheter;
ii) a catheter casing; and
iii) a handle;
wherein the catheter comprises a tube and a funnel, the funnel comprising a handle-engaging portion;
the casing comprises a body having a hollow passage and a lid removably connectable to the body; and
the handle is configured to removably engage with the handle-engaging portion of the catheter funnel.

2. A kit according to claim 1 wherein the lid of the casing is detachable and re-attachable to the body of the casing.

3. A kit according to claim 1 wherein the lid has a flat top.

4. A kit according to claim 3 wherein the interior of the lid is dimensioned to accommodate the bottom of the catheter body, such that the lid can act as a stand for the body.

5. A kit according to claim 1 wherein the body has a flat bottom surface for standing the body in an upright position.

6. A kit according to claim 1 wherein the hollow passage is circular and has a diameter larger than the external diameter of the tube but smaller than the diameter of the funnel.

7. A kit according to claim 1 wherein the lid comprises a hollow internal space which is dimensioned to accommodate the catheter funnel.

8. A kit according to claim 1 wherein the handle-engaging portion of the catheter funnel is formed from a resiliently biased material.

9. A kit according to claim 1 wherein the handle comprises a shaft and a pair of jaws at one end of the shaft to grip the handle-engaging portion of the catheter funnel.

10. A kit according to claim 1 wherein the lid comprises a flat surface on one side and an open end on an opposing side of the flat surface, wherein the open end is dimensioned in order to receive the bottom of the catheter casing body.

11. A kit according to claim 1 wherein an insert is provided within the hollow passage in the catheter casing body.

12. A kit comprising a handle and two or more catheter casings, each containing a catheter, wherein the handle, catheter casings and catheter are as defined in claim 1.

13. A method of inserting a catheter comprising:
i) providing a kit according to claim 1;
ii) engaging the handle with the catheter funnel;
iii) removing the catheter from the catheter casing and inserting the catheter tube into a person (e.g. into the urethra of a patient).

14. A method according to claim 13 further comprising removing the lid from the catheter casing and standing the body of the casing upright in the lid.

15. A method according to claim 13 comprising removing the catheter tube from the person, replacing the catheter into the hollow interior of the catheter casing, detaching the handle from the funnel and replacing the lid onto the body of the catheter casing for safe disposal.
